(19) United States
(12) Patent Application Publication
(10) Pub. No.: US 2022/0233341 A1
(43) Pub. Date: Jul. 28, 2022

(54) Dynamic Metatarsal Roll Bar

(71) Applicant: Instride Shoes, LLC, Hillsborough, NJ (US)

(72) Inventor: Daniel Werremeyer, Hillsborough, NJ (US)

(21) Appl. No.: 17/532,132

(22) Filed: Nov. 22, 2021

Related U.S. Application Data
(60) Provisional application No. 63/141,206, filed on Jan. 25, 2021.

(51) Int. Cl. A61F 5/14; A43B 7/14

(52) U.S. Cl. CPC A61F 5/14; A43B 7/1405

(57) ABSTRACT

A dynamic metatarsal roll bar as a footwear midsole insert, formed of a semi-rigid planar lattice of cells providing flexible support to the forefoot of a user. The cell walls provide structural support, while the planar lattice allows for flexing during a user's stride, returning to a base configuration after pressure from stride movement is relieved.

### PRIORITY

This application claims domestic benefit from pending provisional application no. 63/141,206, filed on Jan. 25, 2021, the contents of which are incorporated by reference.

### FIELD OF INVENTION

The present invention relates to a footwear insert or a footwear midsole with improved support to the foot of a person walking or running.

### BACKGROUND

The human foot is an overly complex mechanism. The many bones, muscles, ligaments, and tendons of the foot function to absorb and dissipate the forces of impact. During running or walking, the foot undertakes several different positions over the course of a single step and is subject to forces 2.5 times the person's body weight.

Metatarsalgia is an inflammatory pain felt mostly in the ball of the foot. It is especially common in women who wear fashion shoes and high heels. Women who have worn this type of footwear in the past are also prone to this type of foot pain, well after ceasing to wear high heeled footwear. Metatarsalgia can also be a problem for men and women who wear regular flat shoes.

Metatarsalgia is caused by the collapsing of the ‘transverse arch’ of the foot. The transverse arch runs across the forefoot and is formed by the five metatarsal bones. A collapse of the transverse arch will lower the transverse arch substantially, in turn putting pressure on the ball of the foot. Many factors can contribute to the collapse or lowering of the transverse arch, including age, muscle weakness, muscle strain, being overweight, wearing improper footwear, high heels, tight footwear, swelling of joints, or other medical conditions.

Untreated metatarsalgia may lead to pain in other parts of the same foot or may cause pain in the opposite foot and or elsewhere in the body. An altered gait from foot pain may also lead to pain in the back or hip.

It is desired to address metatarsalgia, and to provide an improved reduction of several common forefoot conditions including plantar fasciitis, as well as to encourage a more efficient propulsive phase during running and walking.

Lateral flexion of the foot may also cause strain on ankles and knees, as well as increasing pain levels.

Also, there is a present need for a shoe technology that accomplishes the goals of: (1) enhancing the efficiency of the propulsive phase during running and walking, (2) reducing forces on the plantar forefoot; (3) offering graduated support beneath the metatarsals; and (4) providing plantar flexion for active propulsion.

Existing devices used to address metatarsalgia include arch supports and cushioned inserts placed in a shoe beneath the ball of the foot. Arch supports help address the collapse of a transverse arch. Cushioned inserts reduce the impact of a stride with respect to the ball of the foot. However, such existing devices do not provide rigid support against lateral flexing of the foot.

### SUMMARY

A Dynamic Metatarsal Roll Bar is a midsole footwear insert that provides support to a user's foot while the foot is subjected to the forces encountered in running or walking. The Dynamic Metatarsal Roll Bar promotes foot stability by reducing impact forces under the foot, limiting torsional flexing of the shoe, dispersing bottom pressure under the foot and decreasing forefoot flexion. The Dynamic Metatarsal Roll Bar promotes enhanced efficiency of the propulsive phase of walking, running by providing structures which reduce functional hallux limitus (FHL) and off load pressures under the metatarsophalangeal joints.

The Dynamic Metatarsal Roll Bar incorporates a rigid material incapsulated between the midsole and outsole of a shoe, where the rigid material is formed in a roughly planar lattice shape, formed of separate cells, and is located under the metatarsals of the wearer. The Dynamic Metatarsal Roll Bar provides firmer support beneath the metatarsal heads to reduce metatarsal plantar flexion during the propulsive phase of gait. The Dynamic Metatarsal Roll Bar provides support beneath the hallux to reduce hallux dorsiflexion during the propulsive phase of gait. The Dynamic Metatarsal Roll Bar is preferably custom contoured to the shape of the midsole and is lightweight.

### BRIEF DESCRIPTION OF THE DRAWINGS

FIG. 1 is a plan bottom view of the Dynamic Metatarsal Roll Bar.

FIG. 2 is a plan bottom view of the Dynamic Metatarsal Roll Bar, showing its placement within a right shoe midsole.

FIG. 3 is a cross-sectional view of the shoe midsole of FIG. 2, along line A-A of FIG. 2.

FIG. 4 is a cross-sectional view of the shoe midsole of FIG. 2, along line B-B of FIG. 2.

FIG. 5 is x-ray view of the Dynamic Metatarsal Roll Bar as it is encapsulated into the bottom of midsole of a shoe, and located in between the midsole and outsole.

FIG. 6 is an exploded perspective view of an illustrative embodiment of the Dynamic Metatarsal Roll Bar, shoe midsole and shoe outsole.

FIG. 7 is a plan view of three different configurations of the Dynamic Metatarsal Roll Bar, showing its placement within a shoe midsole.

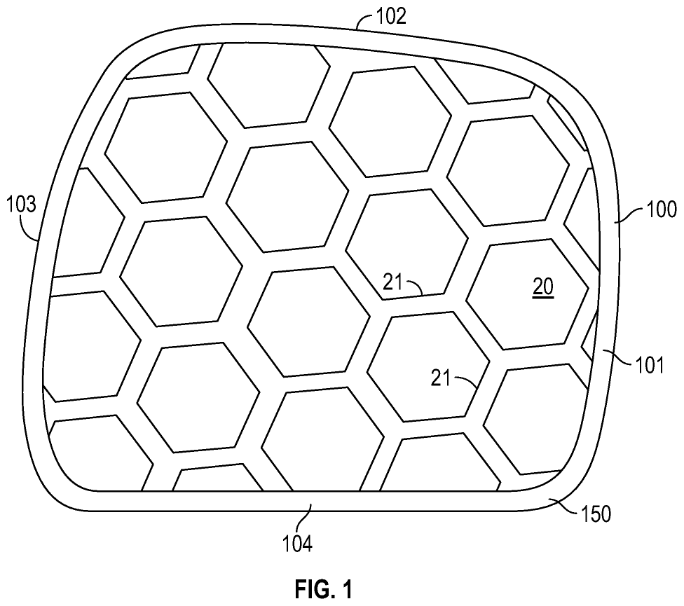

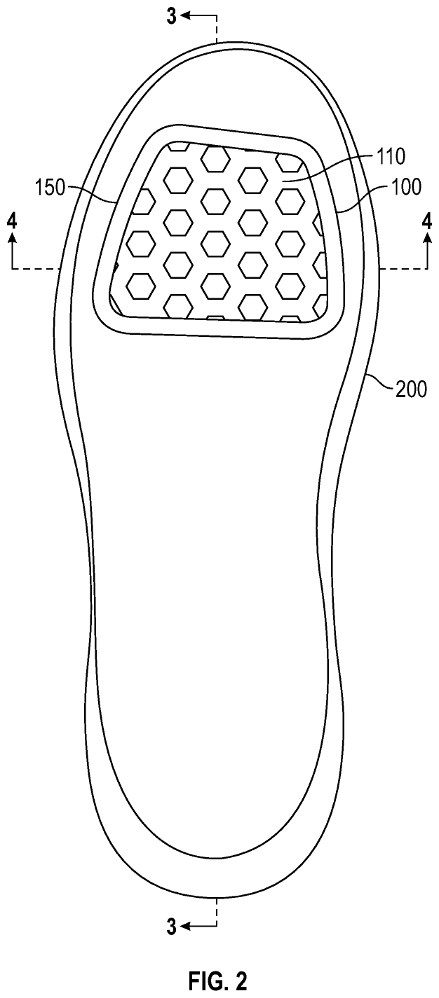

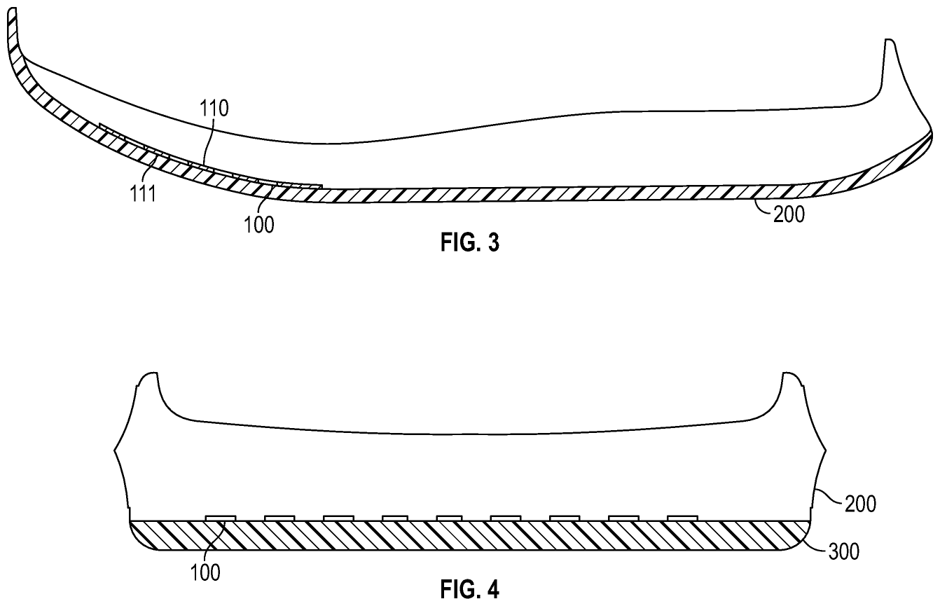

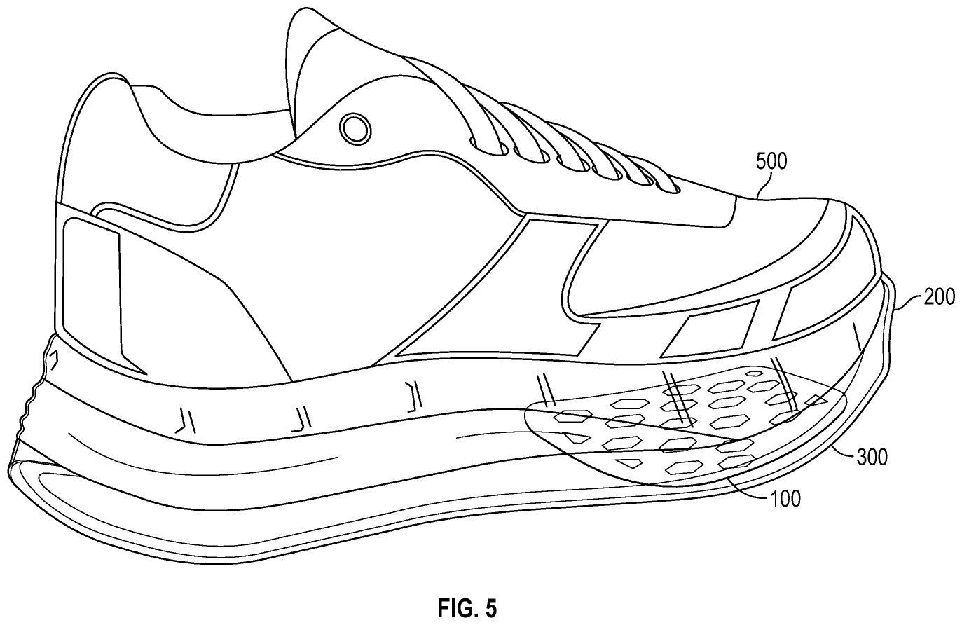

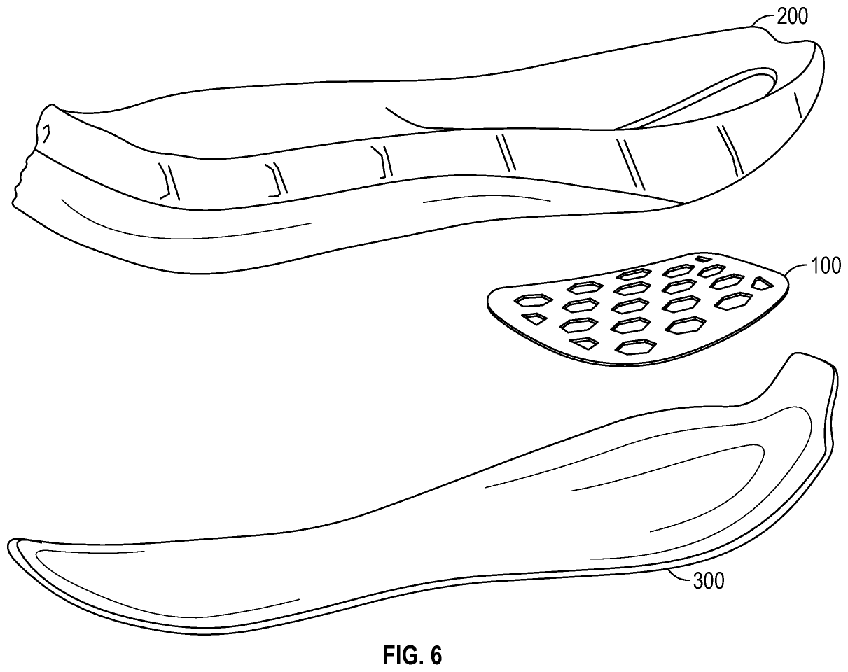

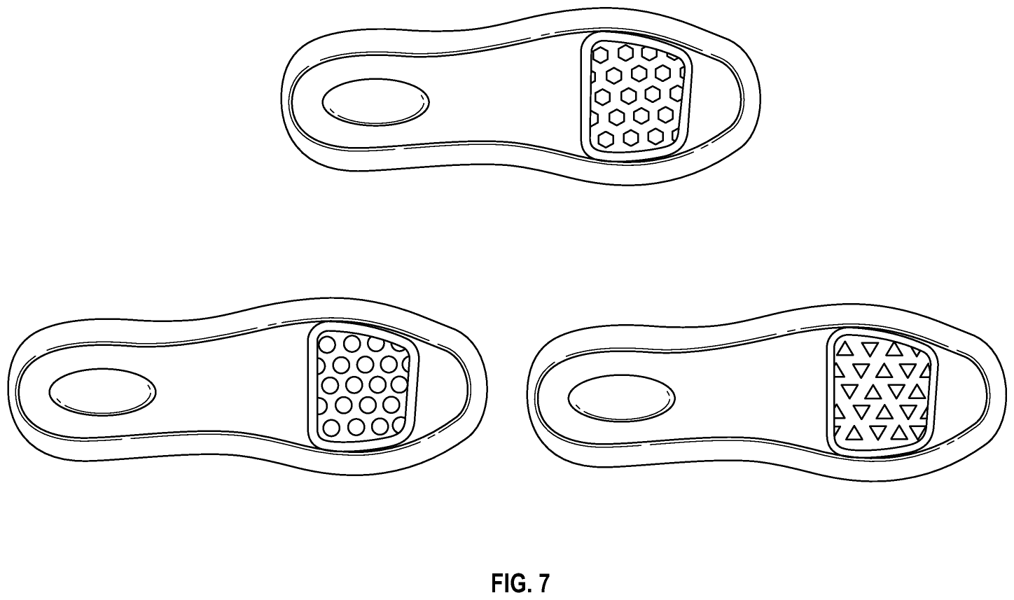

### DETAILED DESCRIPTION

The Dynamic Metatarsal Roll Bar, as a midsole footwear insert, constructed in accordance with the principles of the present invention is disclosed. The Dynamic Metatarsal Roll Bar is used within the midsole of a shoe. As a user's right shoe and left shoe are generally mirror images of one another, the Dynamic Metatarsal Roll Bar for the right shoe and left shoe would be mirror images as well. Only the right side Dynamic Metatarsal Roll Bar is illustrated in the Figures. A left shoe Dynamic Metatarsal Roll Bar is a mirror image construction of the left shoe Dynamic Metatarsal Roll Bar.

This invention helps promote foot stability by reducing impact forces under the foot, providing torsional flexing of the shoe, dispersing bottom pressure under the foot and decreasing forefoot flexion.

The Dynamic Metatarsal Roll Bar 100 is preferably formed as a lattice of a plurality of cells 20, rather than a solid. A lattice of cells 20 provides strength to the Dynamic Metatarsal Roll Bar 100, while keeping the weight to a minimum. The cells 20 of the lattice, each cell 20 formed by cell walls 21, may be of any shape and may not necessarily be of a single shape.

In a preferred embodiment, the Dynamic Metatarsal Roll Bar 100 has a honeycomb structure, with hexagonal cells 20. Such honeycomb structures can be as much as 40 times stronger than a solid of similar material and offer better weight-to-strength ratios. In addition, load carry and load transference qualities are much greater in honeycomb design structure.

The Dynamic Metatarsal Roll Bar 100 is formed in a slightly irregular rectangular or polygonal plane. The Dynamic Metatarsal Roll Bar 100 has roughly linear distal edge 102 and roughly linear proximal edge 104. The Dynamic Metatarsal Roll Bar 100 has a slightly curvilinear medial edge 103 and lateral edge 101, which edges follow, respectively, the curve of the medial and lateral border of the midsole 200 of an item of footwear. The curvilinear medial edge 103 and lateral edge 101 are curved or angled to follow the shape of the midsole 200 and outsole 300 which are usually curvilinear or linear.

Lateral edge 101, distal edge 102, medial edge 103, proximal edge 104 form border 150. Border 150 and the lateral, distal, medial and proximal edges are preferably formed of the same material as cell walls 21. Border 150 surrounds and defines upper surface 110 and lower surface 111.

The Dynamic Metatarsal Roll Bar 100 is positioned under forefoot area, namely the area that correlates with the metatarsal and phalanges of the user's foot. In particular, the Dynamic Metatarsal Roll Bar is positioned with distal edge 102 oriented toward the metatarsophalangeal joint of the foot, proximal edge 104 oriented toward the tarsometatarsal joint of the foot, medial edge 103 oriented toward the medial side of the foot, and lateral edge 101 oriented toward the lateral side of the foot.

The plane of the Dynamic Metatarsal Roll Bar 100 is preferably curved to follow the toe spring of the midsole 200. Upper surface 110 is concave, providing a depression wherein a user's forefoot may be placed when Dynamic Metatarsal Roll Bar is in use. Preferably, lower surface 111 is parallel to upper surface 110, and has a corresponding convex surface.

The Dynamic Metatarsal Roll Bar 100 has a firmer density and is stiffer than the surrounding midsole 200. The difference in density and stiffness is accomplished by using different materials or varying the thickness of the Dynamic Metatarsal Roll Bar 100. The use of different densities and hardness increases the performance of the shoe 500 in handling lateral force impacts and provides improved torsional stability.

The Dynamic Metatarsal Roll Bar can be made in a variety of materials such as carbon fiber, thermoplastic rubber or hard nylon, where the material provides a general rigid structure, resistant to flexing or bending. During a stride, this resistance to flexing provides additional support to the user's foot, especially under the metatarsals. The resistance to flexing prevents the foot from twisting in the middle of the stride.

The Dynamic Metatarsal Roll Bar is preferably between 0.5 mm and 2.5 mm thick, with 1.5 mm thickness being optimal. This thickness provides sufficient torsional rigidity.

In construction, midsole 200 preferably has recessed area to accommodate the Dynamic Metatarsal Roll Bar 100, with a depth matching the thickness of the Dynamic Metatarsal Roll Bar to allow the outsole 300 to be smoothly attached. Encapsulating the

Dynamic Metatarsal Roll Bar 100 in the forefoot area of the midsole 200 reduces the forces against the medial plantar aspect of the heel by decreasing the supination and reducing pronation moments.

When the foot takes up a “neutral” (i.e., neither pronated or supinated) position, the Dynamic Metatarsal Roll Bar 100 applies a gentle forward motion movement while stabilizing movement to the forefoot.

When the foot moves into a pronated position, the Dynamic Metatarsal Roll Bar 100 disperses a higher magnitude of ground forces area beneath the forefoot.

By pronation “control,” we mean the increase in supination moments acting around the joints of the rearfoot and decrease the magnitude of pronation movement and plantarflexion during the propulsive phase of gait.

While certain novel features of the present invention have been shown and described, it will be understood that various omissions, substitutions and changes in the forms and details of the device illustrated and in its operation can be made by those skilled in the art without departing from the spirit of the invention.

I claim:

1. An orthopedic midsole insert comprising:
a planar lattice resistant to torsional force, the planar lattice comprised of
a plurality of cells, where each of the cells comprises a wall surrounding a central cavity, and where adjacent cells share a common wall with one or more of the plurality of cells;
a border surrounding the plurality of cells, the border having a distal edge, a proximal edge, a medial edge, and a lateral edge;
an upper surface;
a lower surface, the upper and lower surfaces formed by the plurality of cells and surrounded by the border; and
where the upper surface of the planar lattice is concave.

2. The orthopedic midsole insert of claim 1, where the wall of each of the plurality of cells is comprised of a plurality of subwalls.

3. The orthopedic midsole insert of claim 2, where the plurality of subwalls form a hexagon shaped cell.

4. The orthopedic midsole insert of claim 1, where the planar lattice is capable of bending in response to force applied thereto, and returns to its original shape after force is no longer applied.

5. The orthopedic midsole insert of claim 1, where the orthopedic midsole insert is incorporated into a midsole of footwear.

6. The orthopedic midsole insert of claim 5, where the planar lattice is located within the forefoot area of the midsole, with the distal edge oriented toward a toe area of the footwear, the proximal edge oriented toward a heel area of the footwear, the medial edge oriented toward the medial side of the footwear, and the lateral edge oriented toward the lateral side of the footwear.

7. An improved footwear midsole, the improvement comprising:
a plurality of cells, where each of the cells comprises a plurality of external edges surrounding a central cavity, and where the plurality of cells are arranged in a planar lattice, with adjacent cells sharing a common edge with one or more of the plurality of cells;
where the planar lattice comprises a continuous border, the border having a distal edge, a proximal edge, a medial edge, and a lateral edge, and where the border is comprised of semi-rigid material; and
where the planar lattice is located in the forefoot area of the footwear midsole.

8. The improved footwear midsole of claim 7, further comprising:
pliable material in the forefoot area of the footwear midsole, where the pliable material is less rigid than at least one of the plurality of cells or border.

9. The improved footwear midsole of claim 8, where the pliable material is located within the cavity of each cell.
